(19) United States
(12) Patent Application Publication
(10) Pub. No.: US 2008/0146910 A1
(43) Pub. Date: Jun. 19, 2008

(54) Non-invasive method for determining body composition using magnetic resonance (MR)

(76) Inventors: Todd Parrish, Evanston, IL (US); Sherif Fahmy, Mount Laurel, NJ (US); Yufen Chen, Chicago, IL (US)

Correspondence Address: Sherif R. Fahmy, 1 Nottingham Way, Mount Laurel, NJ, 08054, US

(21) Appl. No.: 11/944,409

(22) Filed: Nov. 21, 2007

Related U.S. Application Data
(60) Provisional application No. 60/860,588, filed on Nov. 22, 2006.

(51) Int. Cl. A61B 5/055

(52) U.S. Cl. 600/410

(57) ABSTRACT

We present a method for informed optimization of sampling vectors in multi-directional diffusion-weighted magnetic resonance imaging. The advantage of this optimization is that it is informed rather than being a naïve optimization of sampling vectors. Typically, sampling vectors are set relatively uniformly along a spherical surface. In this case, a scan at high imaging resolutions utilizes sampling vectors that are chosen based on the knowledge of the overall orientation distribution for the entire sample or region of interest. This overall orientation distribution is obtained by performing multi-directional diffusion-weighted scans at high angular resolution, but low or minimal voxel resolution. A subset of the vectors used in this high-angular-resolution scan is chosen to minimize the error in the final results. This optimal subset is not necessarily uniform in space.

This application claims the benefit of U.S. Provisional Application No. 60/860,589, filed Nov. 21, 2006

### TITLE OF THE INVENTION: Optimized set of sampling vectors for multi-directional diffusion-weighted magnetic resonance imaging (MRI)

### BACKGROUND OF THE INVENTION

Diffusion-weighted magnetic resonance imaging (DWI) has been used to probe the diffusivity of water molecules in living tissue as well as in other porous media. Any single scan using a diffusion-weighted MRI pulse sequence provides image contrast dependent on the probability of water diffusion along a specific directional axis (the direction being determined by the MRI pulse sequence). Multiple such scans, each providing diffusion sensitization in a different direction and/or with different diffusion weighting, all taken with an additional scan having similar parameters, but without diffusion weighting, may then be analyzed together to extract information about local directional preference of water diffusion in tissue or other media. In order to obtain a orientation distribution as a function of direction in each image voxel (or pixel), as many scans are required as there are directions that we desire to probe, in addition to one similar scan that is not diffusion-weighted (the latter is then simply weighted by the local density of water).

Diffusion-weighted imaging has been used to identify edema and ischemia in living tissue, and has been used in combination with blood-perfusion-weighted imaging to plan intervention in acute stroke. Additionally, changes in measures of diffusion anisotropy (the degree of directional preference for water diffusion) have been used to identify regions of neural degeneration in various neural pathologies. Finally, DWI in multiple directions has been used to track axon bundles and neural connections between different regions in the brain in three dimensions (referred to as “fiber tractography” in the art). This is possible because water diffusion is highly anisotropic (has high directional preference) in fibrous tissue such as neuronal white matter that consists of dense bundles of long tubular neural processes (axons). Using these techniques, studies of anatomical connectivity and changes in connectivity concomitant with pathology enable a better understanding of the functions of different brain centers.

As the richness and accuracy of information on tissue fiber orientation is improved with the number of directions scanned, scan times increase dramatically as researchers and clinicians seek to obtain more information on fiber directions and/or connectivity. As such, many have undertaken to optimize the diffusion scan parameters in order to reduce scan times [1-4]. In general, most of these approaches constitute naïve optimization of the parameters and optimize uniform sampling of the entire diffusion space; however, reference 1 relies on some a priori knowledge of the sample being scanned. We note that reference 1 discusses using known information about sample anisotropy in order to optimize the set of directional scans and other scan parameters. However, our method is more general in that it applies to any arbitrary distribution of fiber orientation.

### DETAILED DESCRIPTION OF THE INVENTION

While the ultimate goal is to obtain a distribution of fiber orientation as a function of angle, for each image voxel (or pixel), we perform a first set of low-resolution or minimal-resolution (i.e. 1 voxel per image) diffusion-weighted scans that sample a large number of orientations, N, that are roughly evenly distributed on a spherical surface. The way the N vectors are distributed may rely on any standard approach in the art [1-4]. This is in order to estimate an accurate overall distribution of fiber orientation as a function of angle in three dimensions, for the entire sample (as opposed to a per-voxel distribution), at high angular sampling density. The sample can be the entire contents of the MR coil or a region limited to excitation by a volume-selective RF pulse. This distribution at high angular density is obtained in a reasonably short time, because only minimal resolution is required. This initial acquisition is a multidirectional DWI acquisition in some N directions.

Next, we choose an optimal set of orientations M that is a small subset of the initial N orientations used. Therefore, M is necessarily less than N. These M orientations, that are significantly fewer than N, are to be used for higher-resolution scans (smaller voxel size). The choice of this optimal set is based on the information obtained from initial low-resolution or minimal-resolution scans. In order to obtain this optimal set of sampling directions (vectors), we use an information-theoretic or other probability-based approach to minimize the error in the final measure desired. This minimization of error is based on the known overall orientation distribution for the whole sample. In one embodiment of the invention, the M sampling vectors are chosen to minimize the sum of per-voxel expected errors in the estimation of the orientation distribution (as a function of direction). This is calculated by noting that the overall orientation distribution for the entire volume is an average of the per-voxel distributions, and by assuming that all configurations that lead to the average distribution are considered equally likely.

In another embodiment of the invention, the sampling vectors are chosen to minimize the worst-case sum of per-voxel errors. Here, we assume the actual sample is a worst-case sample, meaning that its fiber orientations are as unevenly distributed as possible over the region of interest. Thus, we only consider configurations that are as “uneven” as possible, meaning that in these configurations, some voxels are considered “full” in a specific direction. Since the measured diffusion coefficient cannot exceed that of free water, we simply assume it is at this maximal value in these “full” voxels, while the rest of the voxels are assumed empty of fibers along that specific direction. In the most uneven configurations, fibers oriented along any specific direction are assumed to “fill up” as few voxels as possible, while still summing to the overall orientation distribution obtained from the first acquisition, when all the voxels' contributions are summed or averaged. This means that the value of the overall orientation distribution at that specific orientation is assumed to be divided in the most uneven way possible over all the voxels, so that as many voxels as possible contain the maximum diffusion coefficient value at that specific orientation, and the rest are left empty at that specific orientation. The set of such configurations would be considered the set of worst-case configurations (ones that are least realistic for a real sample). The sampling vectors would then be obtained to minimize the error in the worst case. This optimization of sampling vectors would be automated in the scanner, and would not require any computation to be performed by the user, nor any a priori knowledge of the sample. As such, tremendous time savings may be obtained for highly anisotropic media/tissues, with minimal error or minimal loss of information, by using informed optimization, rather than naïve optimization. The time savings would depend on how small we can allow M to be without exceeding the desired error tolerances.

The advantage of this approach should be readily apparent for a highly anisotropic sample, where the overall orientation distribution favors certain directions. The idea is that if M is much less than N, it should provide accuracy still quite high because the M vectors are chosen optimally, in an informed way.

We claim:

1. A method for performing multi-directional diffusion-weighted imaging in magnetic resonance imaging, the method comprising:
Performing a first scan to obtain diffusion-weighted data along a first plurality of sampling vectors over an entire region of interest in the sample or subject being scanned,
Calculating a distribution of fiber orientation for the entire region scanned,
Using this distribution to find an optimal choice of sampling vectors to be used for a second scan, that are a subset of the first plurality of sampling vectors,
Using this optimal choice of sampling vectors to perform a second multidirectional diffusion-weighted scan of the sample or subject, at any higher imaging resolution than the first scan (smaller voxel size).

2. A method according to claim 1, where said optimal choice of sampling vectors is chosen by information-theoretic considerations.

3. A method according to claim 1, where said optimal choice of sampling vectors is chosen by probability-based optimization.

4. A method according to claim 1, where said optimal choice of sampling vectors is chosen to minimize the sum of expected per-voxel errors.

5. A method according to claim 1, where said optimal choice of sampling vectors is chosen to minimize the sum of worst-case per-voxel errors.
